(19) United States
(12) Patent Application Publication
(10) Pub. No.: US 2025/0107759 A1
(43) Pub. Date: Apr. 3, 2025

(54) MONITORING SENSOR RESPONSES TO DIALYSIS

(71) Applicant: Cardiac Pacemakers, Inc., St. Paul, MN (US)

(72) Inventors: John D. Hatlestad, Maplewood, MN (US); Pramodsingh Hirasingh Thakur, Woodbury, MN (US); Craig Stolen, New Brighton, MN (US)

(21) Appl. No.: 18/894,695

(22) Filed: Sep. 24, 2024

Related U.S. Application Data
(60) Provisional application No. 63/541,497, filed on Sep. 29, 2023.

(51) Int. Cl. A61B 5/00; A61B 5/021; A61B 5/053; A61B 7/04; G16H 20/40

(52) U.S. Cl. CPC A61B 5/746; A61B 5/021; A61B 5/053; A61B 5/4836; A61B 5/7264; A61B 5/7275; A61B 7/04; G16H 20/40; A61B 2560/045; A61B 2560/0462; A61B 2562/0204

(57) ABSTRACT

A medical device system includes an ambulatory medical device and a second device. The ambulatory medical device includes one or more physiologic sensors configured to output sensor data and a communication circuit to transmit the sensor data to another device. The second device includes another communication circuit configured to receive the sensor data from ambulatory medical device, and processing circuitry. The processing circuitry is configured to determine patterns of the sensor data consistent with hemodialysis treatments of the patient, identify a change in the patterns of the sensor data indicative of a change in the hemodialysis treatments, and produce an alert associated with the hemodialysis treatments in response to the determining the change in the patterns of the sensor data.

### CLAIM OF PRIORITY

This application claims the benefit of priority under 35 U.S.C. 119(e) of U.S. Provisional Patent Application Ser. No. 63/541,497, filed on Sep. 29, 2023, which is incorporated herein by reference in its entirety.

### TECHNICAL FIELD

This document relates generally to medical devices and more particularly to systems, methods, and devices for automatic monitoring of dialysis of a patient.

### BACKGROUND

Ambulatory medical devices (AMDs), including implantable, subcutaneous, insertable, wearable, or one or more other medical devices, etc., can monitor, detect, or treat, various conditions including, among other things, heart failure (HF), fibrillation, and myocardial infarction. Heart failure is a condition caused by impairment of the blood pumping action of the heart and this can lead to fluid retention by the patient. Heart failure patients may be prescribed hemodialysis (or “dialysis”) to reduce fluid retention. Ambulatory medical devices can include sensors to sense physiological information from a patient and one or more circuits to detect one or more physiologic events using the sensed physiological information. Ambulatory medical devices may also transmit sensed physiologic information or detected physiologic events to one or more remote devices. Patient monitoring with ambulatory medical devices can provide information related to dialysis of patients with HF.

### SUMMARY

Systems and methods are disclosed to monitor the patterns of the dialysis and detect adverse events related to the fluid status of the patient.

In a first Example (Example 1), a method of operating an ambulatory medical device includes obtaining sensor data for a patient using one or more physiologic sensors of the ambulatory medical device, identifying patterns of the sensor data consistent with hemodialysis treatments of the patient, determining a change in the patterns of the sensor data indicative of a change in the hemodialysis treatments, and producing an alert associated with the hemodialysis treatments in response to the determining the change in the patterns of the sensor data.

In Example 2, the subject matter of Example 1 optionally includes sensing physiologic impedance data using an impedance sensor of the ambulatory medical device, identifying a change in the physiologic impedance data indicative of a change in the hemodialysis treatments, and producing the alert using the identified change in the physiologic impedance data.

In Example 3, the subject matter of one or both of Examples 1 and 2 optionally includes sensing heart sound data using a heart sound sensor of the ambulatory medical device, identifying a change in the heart sound data indicative of a change in the hemodialysis treatments, and producing the alert using the identified change in the heart sound data.

In Example 4, the subject matter of Example 3 optionally includes sensing one or both of S3 heart sound data and S4 heart sound data, and producing the alert using an identified change in the one or both of the S3 heart sound data and the S4 heart sound data that is inconsistent with a schedule of hemodialysis treatments of the patient.

In Example 5, the subject matter of Example 3 optionally includes sensing blood pressure data of the patient, sensing S2 heart sound data, and producing an alert regarding risk of fainting using the S2 heart sound data and the blood pressure data.

In Example 6, the subject matter of one or any combination of Examples 1-5 optionally includes sensing physiologic impedance data using an impedance sensor of the ambulatory medical device, identifying a change in the physiologic impedance data indicative of risk of intradialytic hypotension, and producing an alert regarding the risk of intradialytic hypotension in response to identifying the change in the physiologic impedance data.

In Example 7, the subject matter of one or any combination of Examples 1-6 optionally includes sensing physiologic impedance data using an impedance sensor of the ambulatory medical device, and adjusting filtration rate of hemodialysis treatment using the sensed physiologic impedance data.

In Example 8, the subject matter of one or any combination of Examples 1-7 optionally includes comparing, using a separate device that communicates information with the ambulatory medical device, the sensor data for a hemodialysis treatment to sensor data for another hemodialysis treatment, and recommending, by the separate device, a change in dialysis dosage based on the sensor data.

In Example 9, the subject matter of one or any combination of Examples 1-8 optionally includes comparing, using a separate device that communicates information with the ambulatory medical device, the sensor data for a hemodialysis treatment to sensor data for another hemodialysis treatment, and producing, by the separate device, an alert to stop a hemodialysis treatment based on the comparing of the sensor data.

Example 10 includes subject matter (such as a medical device system) or can optionally be combined with one or any combination of Examples 1-9 to include such subject matter, comprising an ambulatory medical device and a second device. The ambulatory medical device includes one or more physiologic sensors configured to output sensor data containing physiological information of a patient, and a communication circuit to transmit the sensor data to another device. The second device includes another communication circuit configured to receive the sensor data from ambulatory medical device and processing circuitry. The processing circuitry of the second device is configured to determine patterns of the sensor data consistent with hemodialysis treatments, identify a change in the patterns of the sensor data indicative of a change in the hemodialysis treatments, and produce an alert associated with the hemodialysis treatments in response to the determining the change in the patterns of the sensor data.

In Example 11, the subject matter of Example 10 optionally includes the ambulatory medical device including an impedance sensor configured to produce physiologic impedance data, and the processing circuitry of the second device configured to identify a change in the physiologic impedance data indicative of a change in the hemodialysis treatments and produce the alert based on the identified change in the physiologic impedance data.

In Example 12, the subject matter of one or both of Examples 10 and 11 optionally includes the ambulatory medical device including a heart sound sensor configured to produce heart sound data, and the processing circuitry of the second device configured to identify a change in the heart sound data indicative of a change in the hemodialysis treatments, and produce the alert based on the identified change in the heart sound data.

In Example 13, the subject matter of Example 12 optionally includes the processing circuitry of the second device configured to identify one or both of S3 heart sounds and S4 heart sounds in the heart sound data, and produce the alert when detecting a change in the one or both of the S3 heart sound and the S4 heart sound that is inconsistent with a schedule of hemodialysis treatments.

In Example 14, the subject matter of Example 12 optionally includes the processing circuitry of the second device configured to receive blood pressure data of the patient, identify S2 heart sounds in the heart sound data, and produce an alert regarding risk of fainting using the S2 heart sound data and the blood pressure data.

In Example 15, the subject matter of one or any combination of Examples 10-14 optionally includes the ambulatory medical device including an impedance sensor configured to produce physiologic impedance data, and the processing circuitry of the second device configured to identify a change in the physiologic impedance data indicative of risk of intradialytic hypotension, and produce an alert regarding the risk of intradialytic hypotension in response to the identifying the change in the physiologic impedance data.

In Example 16, the subject matter of one or any combination of Examples 10-15 optionally includes the processing circuitry of the second device configured to compare the sensor data sensed for a first hemodialysis treatment to sensor data sensed for another hemodialysis treatment, and recommend a change in hemodialysis dosage based on the comparing of the sensor data.

In Example 17, the subject matter of one or any combination of Examples 10-16 optionally includes the processing circuitry of the second device configured to compare the sensor data sensed for a first hemodialysis treatment to sensor data sensed for another hemodialysis treatment, and produce an alert to stop a hemodialysis treatment based on the comparing of the sensor data.

Example 18 includes subject matter (such as an ambulatory medical device) or can optionally be combined with one or any combination of Examples 1-17 to include such subject matter, comprising at least one physiologic sensor configured to produce a sensed physiological signal representative of physiological information of a patient and signal processing circuitry. The signal processing circuitry is configured to identify patterns in the physiological signal consistent with hemodialysis treatments of the patient, determine a change in the identified patterns indicative of a change in the hemodialysis treatments, and produce an alert associated with the hemodialysis treatments in response to the determined change in the patterns of the physiological signal.

In Example 19, the subject matter of Example 18 optionally includes an impedance sensor configured to produce a sensed impedance signal representative of physiological impedance of the patient, and signal processing circuitry configured to identify a change in the sensed impedance signal indicative of a change in the hemodialysis treatments and produce the alert using the identified change in the sensed impedance signal.

In Example 20, the subject matter of one or both of Examples 18 and 19 optionally includes a heart sound sensor configured to produce a heart sound signal representative of mechanical heart activity of the patient, and signal processing circuitry configured to identify a change in the heart sound signal indicative of a change in the hemodialysis treatments and produce the alert using the identified change in the heart sound signal.

These non-limiting Examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example patient management system.

FIG. 2 illustrates an example of an ambulatory medical device (AMD) that is implantable.

FIG. 3 illustrates an example of an AMD that is insertable.

FIG. 4 is a block diagram of an example of electronic circuits of an AMD.

FIG. 5 is a block diagram of an example of electronic circuits of an external device included in a patient management system.

FIG. 6 shows graphs of impedance data obtained by an AMD.

FIGS. 7A-7D are graphs of different sensor data obtained by an AMD.

FIG. 8 is a flow diagram of an example of a method of operating a medical device system.

FIGS. 9A-9C are graphs of sensor data associated with fainting events.

FIG. 10 illustrates a block diagram of an example machine upon which any one or more of the techniques discussed herein may perform.

### DETAILED DESCRIPTION

Ambulatory medical monitoring devices can include, or be configured to receive physiologic information from, one or more sensors located within, on, or proximate to a body of a patient. Physiologic information of the patient can include, among other things, respiration information (e.g., a respiratory rate, a respiration volume (tidal volume), cardiac acceleration information (e.g., cardiac vibration information, pressure waveform information, heart sound information, endocardial acceleration information, acceleration information, activity information, posture information, etc.); impedance information; cardiac electrical information; physical activity information (e.g., activity, steps, etc.); posture or position information; pressure information; plethysmograph information; chemical information; temperature information; or other physiologic information of the patient.

The present inventors have recognized, among other things, that device-based monitoring of a dialysis patient provides valuable physiological information related to the dialysis of the patient. The device-based monitoring can monitor the patterns of the dialysis and may detect adverse events related to the fluid status of the patient.

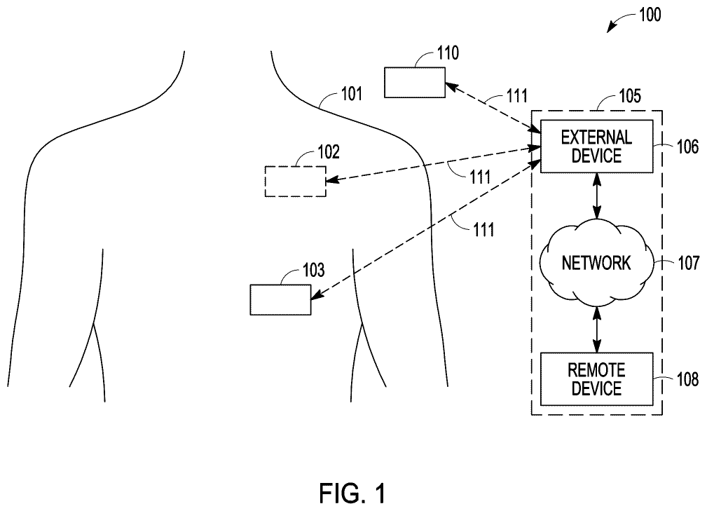

FIG. 1 illustrates an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 can include one or more medical devices, an external system 105, and a communication link 111 providing for communication between the one or more ambulatory medical devices and the external system 105. The one or more medical devices can include an ambulatory medical device (AMD), such as an implantable medical device (IMD) 102, insertable cardiac monitor (ICM), a wearable medical device 103, or one or more other implantable, leadless, subcutaneous, external, wearable, or medical devices configured to monitor, sense, or detect information from, determine physiologic information about, or provide one or more therapies to treat various conditions of the patient 101, such as one or more cardiac or non-cardiac conditions (e.g., dehydration, sleep disordered breathing, etc.).

In an example, the IMD 102 of FIG. 1 can include one or more cardiac rhythm management devices implanted in a chest of a patient, having a lead system including one or more transvenous, subcutaneous, or non-invasive leads or catheters to position one or more electrodes or other sensors (e.g., a heart sound sensor) in, on, or about a heart or one or more other position in a thorax, abdomen, or neck of the patient 101. In another example, the IMD 102 can include a monitor implanted, for example, subcutaneously in the chest of patient 101, the IMD 102 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemakers can communicate between themselves, or one or more other implanted or external devices.

The IMD 102 can include an assessment circuit configured to detect or determine specific physiologic information of the patient 101, or to determine one or more conditions or provide information or an alert to a user, such as the patient 101 (e.g., a patient), a clinician, or one or more other caregivers or processes, such as described herein. The implantable medical device 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy can include delivery of one or more drugs to the patient 101, such as using the implantable medical device 102 or one or more of the other ambulatory medical devices, etc. In some examples, therapy can include CRT for rectifying dyssynchrony and improving cardiac function in heart failure patients. In other examples, the implantable medical device 102 can include a drug delivery system, such as a drug infusion pump to deliver drugs to the patient for managing arrhythmias or complications from arrhythmias, hypertension, hypotension, or one or more other physiologic conditions. In other examples, the implantable medical device 102 can include one or more electrodes configured to stimulate the nervous system of the patient or to provide stimulation to the muscles of the patient airway, etc.

The wearable medical device 103 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 105 can include a dedicated hardware/software system, such as a programmer, a remote server-based patient management system, or alternatively a system defined predominantly by software running on a standard personal computer. The external system 105 can manage the patient 101 through the implantable medical device 102 or one or more other ambulatory medical devices connected to the external system 105 via a communication link 111. In other examples, the IMD 102 can be connected to the wearable medical device 103, or the wearable medical device 103 can be connected to the external system 105, via the communication link 111. This can include, for example, programming the IMD 102 to perform one or more of acquiring physiologic data, performing at least one self-diagnostic test (such as for a device operational status), analyzing the physiologic data, or optionally delivering or adjusting a therapy for the patient 101. Additionally, the external system 105 can send information to, or receive information from, the IMD 102 or the wearable medical device 103 via the communication link 111. Examples of the information can include real-time or stored physiologic data from the patient 101, diagnostic data, such as detection of patient hydration status, hospitalizations, responses to therapies delivered to the patient 101, or device operational status of the implantable medical device 102 or the wearable medical device 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 602.11 wireless fidelity “Wi-Fi” interfacing standards. Other configurations and combinations of patient data source interfacing are possible.

The external system 105 can include an external device 106 in proximity of the one or more ambulatory medical devices, and a remote device 108 in a location relatively distant from the one or more ambulatory medical devices, in communication with the external device 106 via a communication network 107. Examples of the external device 106 can include a medical device programmer. The remote device 108 can be configured to evaluate collected patient or patient information and provide alert notifications, among other possible functions. In an example, the remote device 108 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 108 may additionally include one or more locally configured clients or remote clients securely connected over the communication network 107 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or both of the external device 106 and the remote device 108 can output the detected medical events to a system user, such as the patient or a clinician, or to a process including, for example, an instance of a computer program executable in a microprocessor or other processor. In an example, the process can include an automated generation of recommendations for anti-arrhythmic therapy, or a recommendation for further diagnostic test or treatment. In an example, the external device 106 or the remote device 108 can include a respective display unit for displaying the physiologic or functional signals, or alerts, alarms, emergency calls, or other forms of warnings to signal the detection of arrhythmias. In some examples, the external system 105 can include an external data processor configured to analyze the physiologic or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

The system includes a therapy device 112 that can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 112. The external system 105 can allow for programming the one or more ambulatory medical devices and can receives information about one or more signals acquired by the one or more ambulatory medical devices, such as can be received via a communication link 111. The external system 105 can include a local external implantable medical device programmer. The external system 105 can include a remote patient management system that can monitor patient status or adjust one or more therapies such as from a remote location.

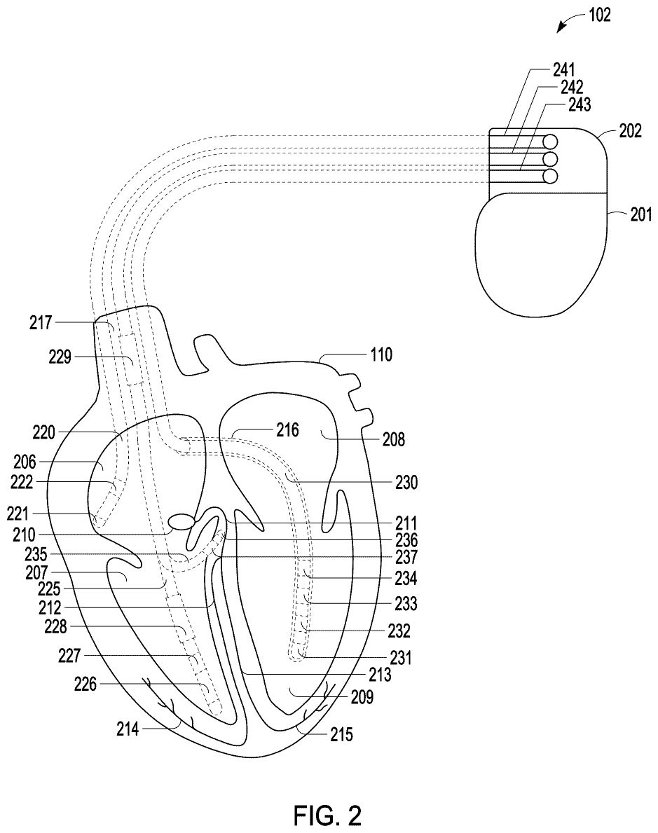

FIG. 2 illustrates an example of an ambulatory medical device that is an IMD 102. The IMD 102 is electrically coupled to a heart 110, such as through one or more leads coupled to the IMD 102 through one or more lead ports, such as first, second, or third lead ports 241, 242, 243 in a header 202 of the IMD 102. In an example, the IMD 102 can include an antenna, such as in the header 202, configured to enable communication with an external system and one or more electronic circuits in a hermetically sealed housing (CAN) 201. The IMD 102 illustrates an example medical device (or a medical device system) as described herein.

The IMD 102 may be an implantable cardiac monitor (ICM), pacemaker, defibrillator, cardiac resynchronizer, or other subcutaneous IMD or cardiac rhythm management (CRM) device configured to be implanted in a chest of a subject, having one or more leads to position one or more electrodes or other sensors at various locations in or near the heart 110, such as in one or more of the atria or ventricles. Separate from, or in addition to, the one or more electrodes or other sensors of the leads, the IMD 102 can include one or more electrodes or other sensors (e.g., a pressure sensor, an accelerometer, a gyroscope, a microphone, etc.) powered by a power source in the IMD 102. The one or more electrodes or other sensors of the leads, the IMD 102, or a combination thereof, can be configured detect physiologic information from, or provide one or more therapies or stimulation to, the patient.

The IMD 102 can include one or more electronic circuits configured to sense one or more physiologic signals, such as an electrogram or a signal representing mechanical function of the heart 110. In certain examples, the CAN 201 may function as an electrode such as for sensing or pulse delivery. For example, an electrode from one or more of the leads may be used together with the CAN 201 such as for unipolar sensing of an electrogram or for delivering one or more pacing pulses. A defibrillation electrode (e.g., the first defibrillation coil electrode 228, the second defibrillation coil electrode 229, etc.) may be used together with the CAN 201 to deliver one or more cardioversion/defibrillation pulses.

The example lead configurations in FIG. 2 include first, second, and third leads 220, 225, 230 in traditional lead placements in the right atrium (RA) 206, right ventricle (RV) 207, and in a coronary vein 216 (e.g., the coronary sinus) over the left atrium (LA) 208 and left ventricle (LV) 209, respectively, and a fourth lead 235 positioned in the RV 207 near the His bundle 211, between the AV node 210 and the right and left bundle branches 212, 213 and Purkinje fibers 214, 215. Each lead can be configured to position one or more electrodes or other sensors at various locations in or near the heart 110 to detect physiologic information or provide one or more therapies or stimulation.

The first lead 220, positioned in the RA 206, includes a first tip electrode 221 located at or near the distal end of the first lead 220 and a first ring electrode 222 located near the first tip electrode 221. The second lead 225 (dashed), positioned in the RV 207, includes a second tip electrode 226 located at or near the distal end of the second lead 225 and a second ring electrode 227 located near the second tip electrode 226. The third lead 230, positioned in the coronary vein 216 over the LV 209, includes a third tip electrode 231 located at or near the distal end of the third lead 230, a third ring electrode 232 located near the third tip electrode 231, and two additional electrodes 233, 234. The fourth lead 235, positioned in the RV 207 near the His bundle 211, includes a fourth tip electrode 236 located at or near the distal end of the fourth lead 235 and a fourth ring electrode 237 located near the fourth tip electrode 236. The tip and ring electrodes can include pacing/sensing electrodes configured to sense electrical activity or provide pacing stimulation.

In addition to tip and ring electrodes, one or more leads can include one or more defibrillation coil electrodes configured to sense electrical activity or provide cardioversion or defibrillation shock energy. For example, the second lead 225 includes a first defibrillation coil electrode 228 located near the distal end of the second lead 225 in the RV 207 and a second defibrillation coil electrode 229 located a distance from the distal end of the second lead 225, such as for placement in or near the superior vena cava (SVC) 217.

Different CRM devices include different number of leads and lead placements. For examples, some CRM devices are single-lead devices having one lead (e.g., RV only, RA only, etc.). Other CRM devices are multiple-lead devices having two or more leads (e.g., RA and RV; RV and LV; RA, RV, and LV; etc.). CRM devices adapted for His bundle pacing often use lead ports designated for LV or RV leads to deliver stimulation to the His bundle 211.

The IMD 102 can include one or more electronic circuits configured to sense impedance between electrodes. In an example, the IMD 102 can sense impedance such as between electrodes located on one or more of the leads or the CAN 201. The IMD 102 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc.

The impedance signal can represent different physiologic impedances of the patient depending on the position of the electrodes used in determining the impedance. In an example, the IMD 102 can be configured to inject current between an electrode on one or more of the first, second, third, or fourth leads 220, 225, 230, 235 and the CAN 201, and to sense the resultant voltage between the same or different electrodes and the CAN 201. The impedance of the measurement is across a significant portion of the thorax region of the patient (e.g., thoracic impedance). In another example, the IMD 102 can sense impedance using a bipolar configuration (e.g., between ring electrode 227 and defibrillation coil electrode 228) to measure impedance within a chamber of the heart (e.g., intracardiac impedance).

The IMD 102 can include a heart sound sensor to produce a heart sound signal. Heart sounds are recurring mechanical signals associated with cardiac vibrations or accelerations from blood flow through the heart or other cardiac movements with each cardiac cycle or interval and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4).

Valve closures and blood movement and pressure changes in the heart can cause accelerations, vibrations, or movement of the cardiac walls that can be detected using a heart sound sensor such as an accelerometer or a microphone, producing a heart sound signal. In an example, heart sound signal portions, or values of respective heart sound signals for a cardiac interval, may be detected by comparison with a sensed cardiac signal. For instance, the value and timing of an S1 signal can be detected using an amplitude or energy of the heart sound signal occurring at or about the R wave of the cardiac interval. The S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles. For example, a heart sound parameter can include a composite S1 parameter representative of a plurality of S1 parameters, for example, over a certain time period (e.g., a number of cardiac cycles, a representative time period, etc.). In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform.

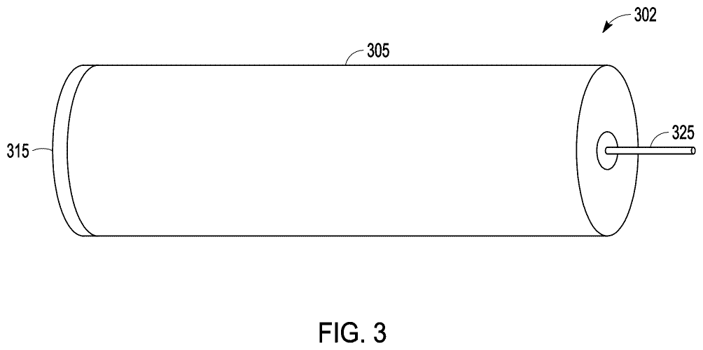

FIG. 3 illustrates portions of an example of a leadless ambulatory medical device (AMD) 302 that is insertable. In some examples, the AMD 302 is an insertable cardiac monitoring device (ICM). The AMD 302 includes a housing having an elongate cylindrical body 305. The AMD 302 can include one or more physiologic sensors to collect physiological information of the patient. In some examples, AMD includes a cardiac signal sensing circuit. The cardiac signal sensor produces sensed cardiac signals when connected to electrodes. The AMD 302 can include a first electrode incorporated into a pin 325 arranged at a first capped end of the housing, and a second electrode incorporated into a surface located at a second capped end 315. Portions of the pin 325 and the surface of the second capped end 315 are conductive and contact tissue and fluid. The cylindrical housing is electrically insulating. The cylindrical housing can include a ceramic or plastic. The electrodes can be used for both sensing and communication. In some examples, the electrodes located at the ends of AMD 302 are used for communication and a separate set of electrodes is included on the housing for cardiac signal sensing.

In some examples, the AMD 302 can include a heart sound sensor within the housing, such as an accelerometer or a microphone. In some examples, the AMD 302 can include an activity sensor such as an accelerometer or a tilt switch to detect activity such as movement of the patient. In some examples, the AMD 302 includes an impedance sensor that produces an impedance signal representative of impedance between the electrodes of the AMD 302.

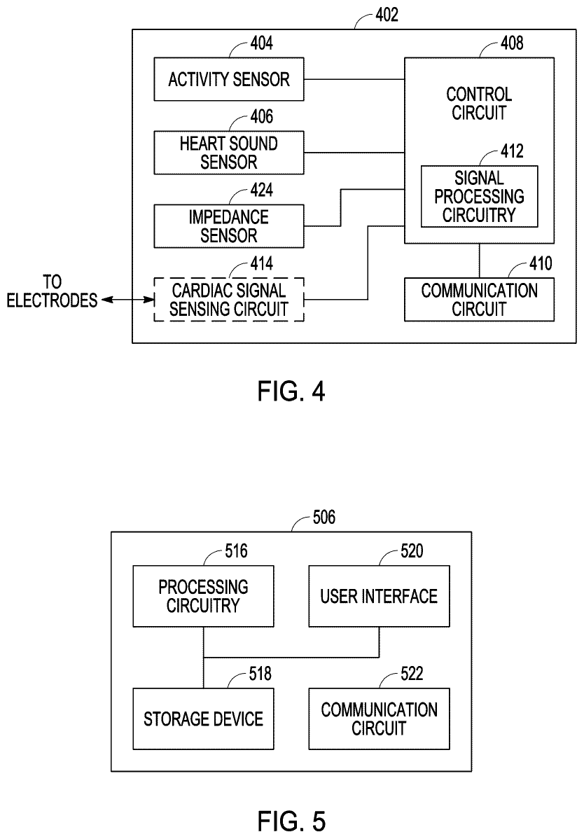

FIG. 4 is a block diagram of an example of portions of electronic circuits of an AMD 402. The AMD 402 may be implantable or insertable. The AMD 402 includes an activity sensor 404, a heart sound sensor 406, an impedance sensor 424, a communication circuit 410, and a control circuit 408. The activity sensor 404 produces an activity signal representative of activity level of a patient, and the heart sound sensor 406 produces a heart sound signal that include electrical representations of heart sounds of the patient. The impedance sensor produces an impedance signal representative of electrical impedance of the patient. The communication circuit 410 can be used to communicate information wirelessly with a separate device, such as by using near-field inductive wireless signals or far-field radio-frequency signals.

The control circuit 408 may include a digital signal processor, application specific integrated circuit (ASIC), field programmable gate array (FPGA), microprocessor, or other type of processor, interpreting or executing instructions in software or firmware. In some examples, the control circuit 408 may include a state machine or sequencer that is implemented in hardware circuits. The control circuit 408 may include any combination of hardware, firmware, or software. The control circuit 408 includes electronic circuitry (e.g., signal processing circuitry 412) to perform the functions described herein. A circuit may include software, hardware, firmware, or any combination thereof. For example, the circuit may include instructions in software executing on the control circuit 408. Multiple functions may be performed by one or more circuits of the control circuit 308. The AMD 402 may include other physiologic sensors such as a cardiac signal sensing circuit 414 for example.

FIG. 5 is a block diagram of an example of portions of a device 506 included in an external system (e.g., external system 105 in FIG. 1). The device 506 may be either an external device of the external system (e.g., external device 106 in FIG. 1) or a remote device of the external system (e.g., remote device 108 in FIG. 1). The device 506 includes a storage device 518 and processing circuitry 516. In some examples, the device 506 includes a user interface 520. Processing circuitry 516 may be implemented using an application-specific integrated circuit (ASIC) constructed to perform one or more functions or a general-purpose circuit programmed to perform the functions. A general-purpose circuit can include, among other things, a microprocessor or a portion thereof, a microcontroller or portions thereof, and a programmable logic circuit or a portion thereof. The storage device 518 may be a memory integral to the processing circuitry 516, or a separate memory device.

The device 506 includes a communication circuit 522 to communicate information with another device. Communication circuit 522 may communicate information wirelessly with the AMD 402 of FIG. 4 using near-field inductive wireless signals or far-field radio-frequency signals. The device 506 can be used to program pacing therapy parameters and other information in the AMD 402. In some examples, the device 506 is remote from the AMD 402 and the communication circuit 522 communicates information over a network (e.g., a cellular phone network or the Internet) to another device that communicates information wirelessly with the AMD 402.

Patients with HF may experience excessive fluid buildup and may be prescribed dialysis as part of management of their fluid status. Physiologic sensors (such as the sensors of an AMD) may provide sensor data useful in tracking the dialysis of the patient. Dialysis is typically performed three times per week and dialysis sessions typically last four hours. For example, common schedules are four-hour dialysis sessions on each of Monday, Wednesday, and Friday during the week, or on Tuesday. Thursday, and Saturday. By performing synchronous averaging over days and hours of patient sensor data from ambulatory medical devices (e.g., implanted devices), patterns in the sensor data may show differences on days where the patient undergoes hemodialysis.

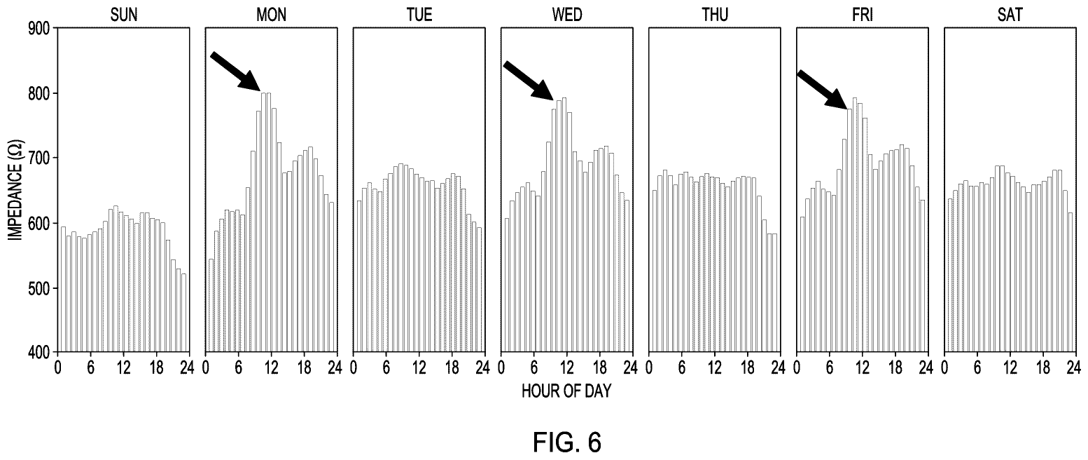

FIG. 6 shows graphs of impedance data obtained by an ICM for a patient undergoing dialysis treatment. The impedance data was obtained by an impedance sensor of an ambulatory medical device. The graphs represent hundreds of days of impedance data for the patient. The arrows in the graphs indicate when the hours of dialysis occurred near noon on Mondays, Wednesdays, and Fridays. The impedance data shows peaks in measured impedance during the dialysis. The peaks in impedance may be due to reducing patient fluid during the dialysis treatments. Thus, the impedance data may be used to track the dialysis of the patient. Sensor data from other sensors show the changes due to dialysis treatments.

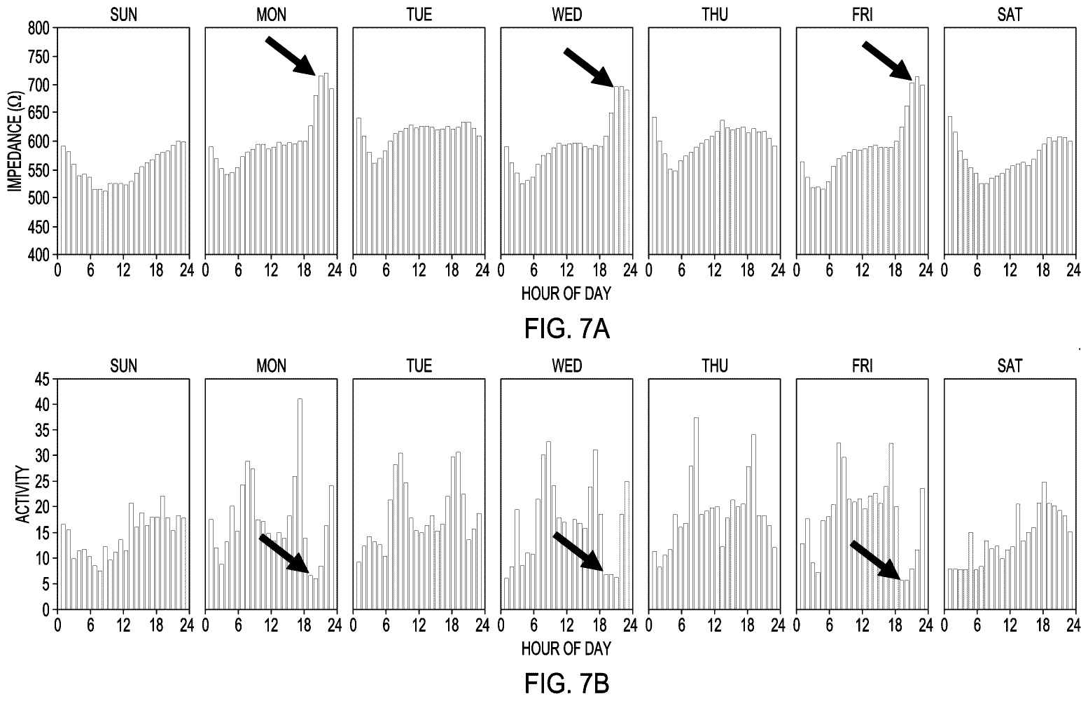

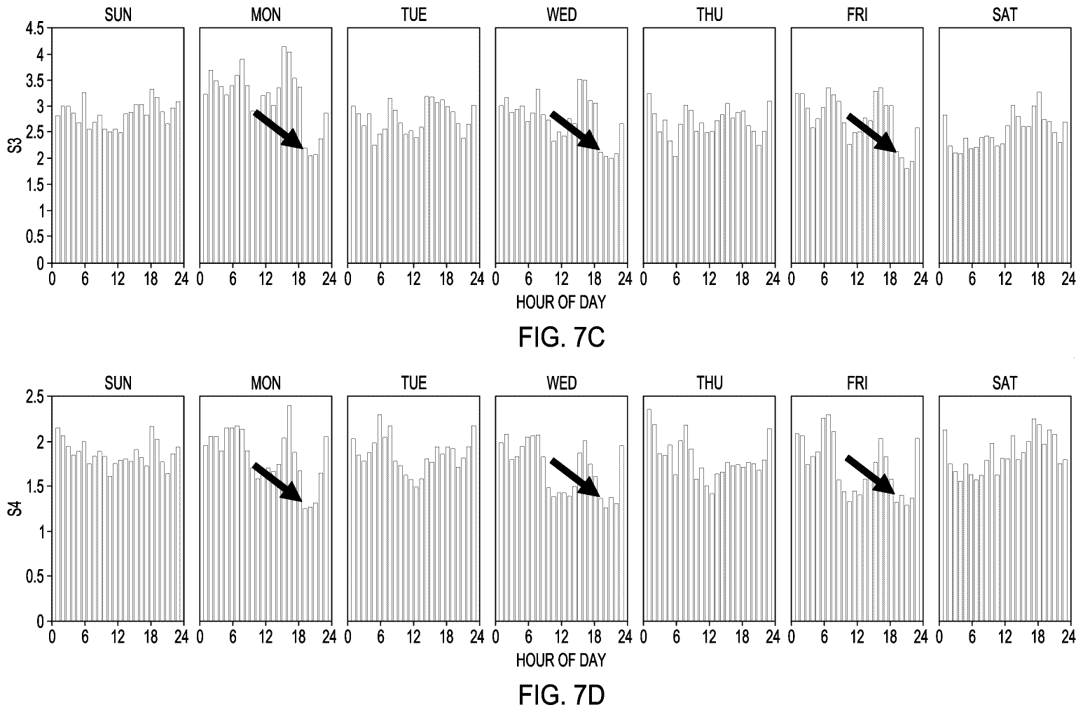

FIGS. 7A-7D are graphs of different sensor data obtained by an ICM for another patient undergoing dialysis treatment. FIG. 7A are graphs on impedance data. The arrows in the graphs indicate when the hours of dialysis occurred prior to midnight on Mondays, Wednesdays, and Fridays. As in FIG. 6, the impedance data in FIG. 7A shows peaks in measured impedance during the dialysis. FIG. 7B are graphs of activity data for the patient. The activity data was obtained by an activity sensor of an ambulatory medical device. The graphs in FIG. 7B show a decrease in activity of the patient during the dialysis treatments, as would be expected for a patient connected to a dialysis machine. The times of lower activity of the patient track the times of higher impedance in FIG. 7A.

FIGS. 7C and 7D are graphs of S3 heart sound data and S4 heart sound data respectively. The heart sound data was obtained by a heart sound sensor of an ambulatory medical device. The arrows in the graphs of FIGS. 7C and 7D show a decrease in S3 and S4 heart sounds associated with the dialysis treatments. As noted previously herein, the S3 and S4 heart sounds are associated with increased filling pressures of the left ventricle. The decrease in the heart sounds with dialysis may be due to decreased filling pressures due to the decreased amount of fluid result from the dialysis treatments. The graphs of sensor data of FIGS. 6 and 7A-7D show that sensor data obtained by ambulatory medical devices can be used to monitor the weekday dialysis patterns of a patient.

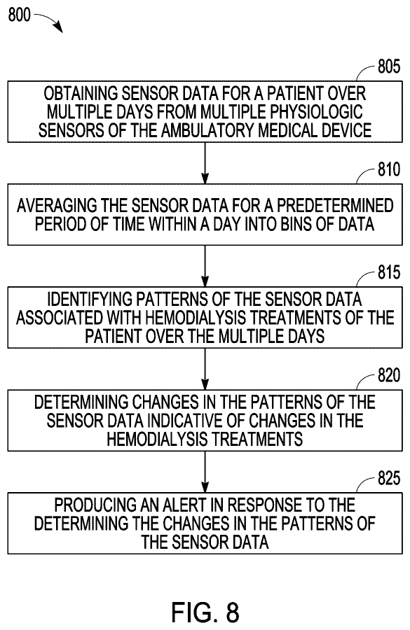

FIG. 8 is a flow diagram of an example of a method 800 of operating a medical device system (e.g., the patient management system 100 of FIG. 1). The medical device system includes an ambulatory medical device, such as any of the ambulatory medical devices described herein. At block 805, sensor data is obtained for a patient. The data may be taken over multiple days. The sensor data is obtained from multiple physiologic sensors of the ambulatory medical device.

At block 810, the sensor data obtained for a predetermined time period within a day is averaged into bins of sensor data. For example, sensor data is obtained for a patient over the course of a week. The sensor data for each hour of the day is averaged and binned into hourly bins within each weekday. The sensor data may be averaged by the ambulatory medical device, or in certain examples, the sensor data is sent to an external system (e.g., external system 105 in FIG. 1) which averages the sensor data into the bins.

At block 815, patterns of the sensor data are identified. The patterns in the sensor data are associated with hemodialysis treatments of the patient over the multiple days. The bins may show patterns in the dialysis of the patent for each day of the week (e.g., the patterns described regarding FIGS. 6 and 7A-7B). The patterns shown by the sensor data may be used to identify if the patterns in the sensor data are consistent with dialysis sessions.

At block 820, changes in the identified patterns are determined that are indicative of changes in the hemodialysis treatments. Changes in the patterns with time may indicate changes in the dialysis treatments of the patient with time. These changes may occur if the patient is omitting, or otherwise not receiving, the dialysis treatments. At block 825, an alert is produced in response to the determining the changes in the patterns of the sensor data.

The changes in the patterns may be identified by the ambulatory medical device or the external system. The processing circuitry of the ambulatory medical device or the external system may perform machine learning algorithms to identify changes in sensor signals obtained by multiple physiologic sensors to identify the changes in dialysis treatments. If the patient is a heart failure patient, the external system may identify changes in treatment and alert the patient's heart failure physician that there are changes in the dialysis treatments that may affect the patient's medical condition.

In some examples, the external system may automatically produce a recommendation regarding the dialysis dosage for the patient (e.g., frequency and duration of dialysis treatments, or how much fluid to remove during a session). For instance, the external system may receive information regarding adverse events for the patient for dialysis sessions. As shown in FIGS. 6 and 7A-7B, the sensor data changes during the dialysis treatment. The changes in sensor data for dialysis sessions with no adverse events reported can be used to make a recommendation of dialysis dosage. For instance, the external system may produce a recommendation to stop the dialysis dosage when the sensor data for the current dialysis session reaches the sensor data values matching or within a specific range of the sensor data for sessions with no adverse events reported.

In another example, changes in both of sensor data for dialysis sessions with no adverse events reported and sensor data for dialysis sessions with adverse events reported can be used to make a recommendation on dialysis dosage. Initial values of the sensor data at the start of the current dialysis treatment can be compared to the initial values of sensor data of previous dialysis treatments. The external system may recommend stopping the current session when the sensor data reaches values similar to the final values of past sessions with no adverse events reported, or when the sensor data reaches values better for the patient than the sensor data for past sessions with adverse events reported (e.g., lower impedance values or higher amplitude heart sounds).

In some examples, the external system may predict intradialytic hypotension (IDH). Fluid extraction by ultrafiltration results in a sudden fluid compartment change that causes blood pressure instability. The ultrafiltration rate (UFR) is a key predisposing factor to IDH, especially when it exceeds the plasma refill rate, with the risk for IDH increasing greatly with increasing gaps between UFR and plasma refill. The rapid loss of volume overwhelms the compensatory mechanisms and the plasma refilling and venous return lag behind. Autonomic dysfunction or decreased contractility disrupt the compensatory mechanisms even further, thus patients with chronic heart failure (CHF) tend to develop IDH with lower UFRs.

Transient gaps in impedance measured by the ambulatory medical device, concurrent with hemodialysis, may be associated with gaps between UFR and plasma refill. The transient gaps in impedance may represent fluid compartment changes and thus may be a marker of IDH risk for the dialysis patient. The external system may monitor impedance information from the ambulatory medical device and may generate an alert when transient gaps in impedance are detected to warn of IDH risk. A feedback loop of impedance information may be used to control UFR to prevent sudden fluid compartment changes.

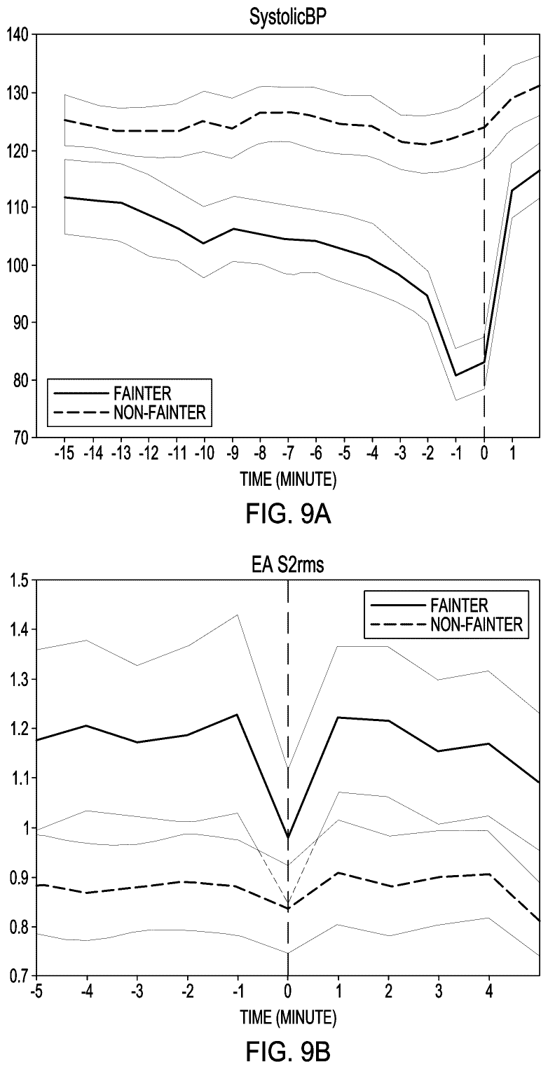

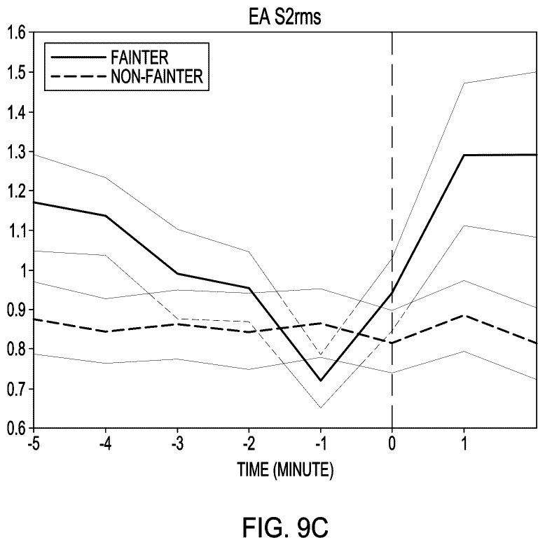

Patients undergoing hemodialysis that have IDH may require special attention after dialysis because of the possibility of fainting. Sensor data collected by the ambulatory medical device may be used to predict fainting by the dialysis patient. FIG. 9A is a graph of systolic blood pressure (BP) versus time for two dialysis patients. One patient experienced a fainting event during dialysis and the other patient did not experience a fainting incident. The fainting event is indicated in the graph as time “0” and the graph shows the sudden decrease in blood pressure associated with the fainting event. The graph shows that the blood pressure was lower for the fainting patient prior to the fainting event.

FIGS. 9B and 9C are graphs of the S2 heart sound amplitude for patients who experienced a fainting event and patients who did not experience a fainting event. The fainting event is again indicated as time “0” in the graphs. In the graphs, the amplitude of the S2 heart sound was higher for the fainting patients prior to the fainting event than for the non-fainting patients. The graphs show that blood pressure information and S2 heart sound information can be predictive of fainting by the dialysis patient, such as when the patient has low blood pressure combined with higher amplitude S2 heart sounds. The external system may receive S2 heart sound information from the ambulatory medical device and receive blood pressure information from another device. The external system may generate an alert of risk of fainting by the patient using the S2 heart sound information and the blood pressure information.

The several examples of systems, devices, and methods described herein show that sensor data obtained by a monitoring device can provide information helpful in tracking the hemodialysis of patients. Device-based monitoring can provide alerts to changes in the dialysis and can provide recommendations regarding changes in the dialysis.

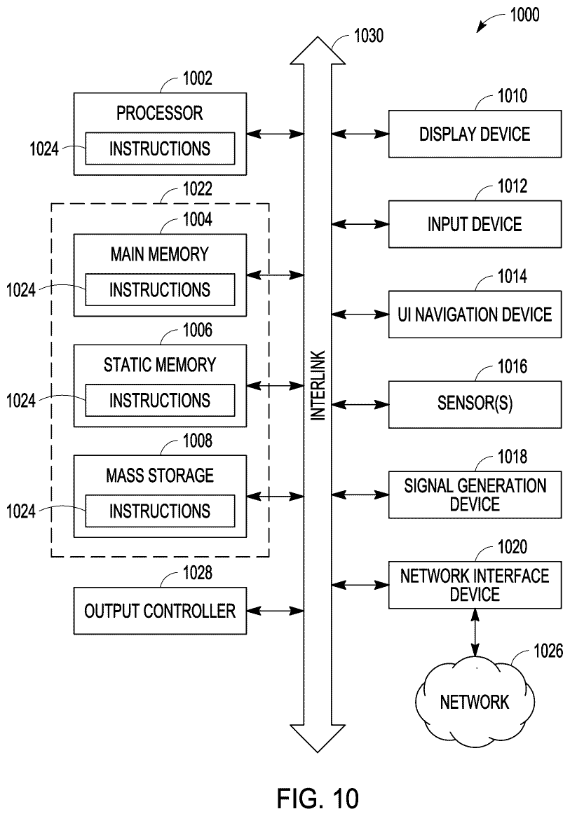

FIG. 10 is a block diagram of an example machine 1000 upon which any one or more of the techniques (e.g., methodologies) discussed herein may perform. Portions of this description may apply to the computing framework of one or more of the medical devices described herein, such as the ambulatory medical device, the wearable medical device, the external programmer, the remoter server, etc., in computing a composite health index. Further, as described herein with respect to medical device components, systems, or machines, such may require regulatory-compliance not capable by generic computers, components, or machinery.

Examples, as described herein, may include, or may operate by, logic or a number of components, or mechanisms in the machine 1000. Circuitry (e.g., signal processing circuitry, etc.) is a collection of circuits implemented in tangible entities of the machine 1000 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1000 follow.

In alternative embodiments, the machine 1000 may operate as a standalone device or may be connected (e.g., networked) to other machines. In a networked deployment, the machine 1000 may operate in the capacity of a server machine, a client machine, or both in server-client network environments. In an example, the machine 1000 may act as a peer machine in peer-to-peer (P2P) (or other distributed) network environment. The machine 1000 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 1000 (e.g., computer system) may include a hardware processor 1002 (e.g., a central processing unit (CPU), a graphics processing unit (GPU), a hardware processor core, or any combination thereof), a main memory 1004, a static memory 1006 (e.g., memory or storage for firmware, microcode, a basic-input-output (BIOS), unified extensible firmware interface (UEFI), etc.), and mass storage 1008 (e.g., hard drive, tape drive, flash storage, or other block devices) some or all of which may communicate with each other via an interlink 1030 (e.g., bus). The machine 1000 may further include a display unit 1010, an input device 1012 (e.g., a keyboard), and a user interface (UI) navigation device 1014 (e.g., a mouse). In an example, the display unit 1010, input device 1012, and UI navigation device 1014 may be a touch screen display. The machine 1000 may additionally include a signal generation device 1018 (e.g., a speaker), a network interface device 1020, and one or more sensors 1016, such as a global positioning system (GPS) sensor, compass, accelerometer, or one or more other sensors. The machine 1000 may include an output controller 1028, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the hardware processor 1002, the main memory 1004, the static memory 1006, or the mass storage 1008 may be, or include, a machine-readable medium 1022 on which is stored one or more sets of data structures or instructions 1024 (e.g., software) embodying or utilized by any one or more of the techniques or functions described herein. The instructions 1024 may also reside, completely or at least partially, within any of registers of the hardware processor 1002, the main memory 1004, the static memory 1006, or the mass storage 1008 during execution thereof by the machine 1000. In an example, one or any combination of the hardware processor 1002, the main memory 1004, the static memory 1006, or the mass storage 1008 may constitute the machine-readable medium 1022. While the machine-readable medium 1022 is illustrated as a single medium, the term “machine-readable medium” may include a single medium or multiple media (e.g., a centralized or distributed database, and/or associated caches and servers) configured to store the one or more instructions 1024.

The term “machine-readable medium” may include any medium that is capable of storing, encoding, or carrying instructions for execution by the machine 1000 and that cause the machine 1000 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 1024 may be further transmitted or received over a communications network 1026 using a transmission medium via the network interface device 1020 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®. IEEE 802.16 family of standards known as WiMax®). IEEE 802.15.4 family of standards, peer-to-peer (P2P) networks, among others. In an example, the network interface device 1020 may include one or more physical jacks (e.g., Ethernet, coaxial, or phone jacks) or one or more antennas to connect to the communications network 1026. In an example, the network interface device 1020 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 1000, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A method of operating an ambulatory medical device, the method comprising:
obtaining sensor data for a patient using one or more physiologic sensors of the ambulatory medical device;
identifying patterns of the sensor data consistent with hemodialysis treatments of the patient;
determining a change in the patterns of the sensor data indicative of a change in the hemodialysis treatments; and
producing an alert associated with the hemodialysis treatments in response to the determining the change in the patterns of the sensor data.

2. The method of claim 1, including:
sensing physiologic impedance data using an impedance sensor of the ambulatory medical device;
identifying a change in the physiologic impedance data indicative of a change in the hemodialysis treatments; and
producing the alert using the identified change in the physiologic impedance data.

3. The method of claim 1, including:
sensing heart sound data using a heart sound sensor of the ambulatory medical device;
identifying a change in the heart sound data indicative of a change in the hemodialysis treatments; and
producing the alert using the identified change in the heart sound data.

4. The method of claim 3,
wherein the sensing heart sound data includes sensing one or both of S3 heart sound data and S4 heart sound data; and
wherein the producing the alert includes producing the alert using an identified change in the one or both of the S3 heart sound data and the S4 heart sound data that is inconsistent with a schedule of hemodialysis treatments of the patient.

5. The method of claim 3, including:
sensing blood pressure data of the patient;
wherein the sensing heart sound data includes sensing S2 heart sound data; and
wherein the producing the alert includes producing an alert regarding risk of fainting using the S2 heart sound data and the blood pressure data.

6. The method of claim 1, including:
sensing physiologic impedance data using an impedance sensor of the ambulatory medical device;
identifying a change in the physiologic impedance data indicative of risk of intradialytic hypotension; and
producing an alert regarding the risk of intradialytic hypotension in response to identifying the change in the physiologic impedance data.

7. The method of claim 1, including:
sensing physiologic impedance data using an impedance sensor of the ambulatory medical device; and
adjusting filtration rate of hemodialysis treatment using the sensed physiologic impedance data.

8. The method of claim 1, including:
comparing, using a separate device that communicates information with the ambulatory medical device, the sensor data for a hemodialysis treatment to sensor data for another hemodialysis treatment; and
recommending, by the separate device, a change in dialysis dosage based on the sensor data.

9. The method of claim 1, including:
comparing, using a separate device that communicates information with the ambulatory medical device, the sensor data for a hemodialysis treatment to sensor data for another hemodialysis treatment; and
wherein the producing the alert includes producing, by the separate device, an alert to stop a hemodialysis treatment based on the comparing of the sensor data.

10. A medical device system, the system comprising:
an ambulatory medical device including:
one or more physiologic sensors configured to output sensor data containing physiological information of a patient; and
a communication circuit to transmit the sensor data to another device;
a second device including:
another communication circuit configured to receive the sensor data from ambulatory medical device; and
processing circuitry configured to:
determine patterns of the sensor data consistent with hemodialysis treatments;
identify a change in the patterns of the sensor data indicative of a change in the hemodialysis treatments; and
produce an alert associated with the hemodialysis treatments in response to the determining the change in the patterns of the sensor data.

11. The system of claim 10,
wherein the ambulatory medical device includes an impedance sensor configured to produce physiologic impedance data;
wherein the processing circuitry of the second device is configured to:
identify a change in the physiologic impedance data indicative of a change in the hemodialysis treatments; and
produce the alert based on the identified change in the physiologic impedance data.

12. The system of claim 10,
wherein the ambulatory medical device includes a heart sound sensor configured to produce heart sound data;
wherein the processing circuitry of the second device is configured to:
identify a change in the heart sound data indicative of a change in the hemodialysis treatments; and
produce the alert based on the identified change in the heart sound data.

13. The system of claim 12,
wherein processing circuitry of the second device is configured to:
identify one or both of S3 heart sounds and S4 heart sounds in the heart sound data;
produce the alert when detecting a change in the one or both of the S3 heart sound and the S4 heart sound that is inconsistent with a schedule of hemodialysis treatments.

14. The system of claim 12,
wherein the processing circuitry of the second device is configured to:
receive blood pressure data of the patient;
identify S2 heart sounds in the heart sound data; and
produce an alert regarding risk of fainting using the S2 heart sound data and the blood pressure data.

15. The system of claim 10,
wherein the ambulatory medical device includes an impedance sensor configured to produce physiologic impedance data;
wherein the processing circuitry of the second device is configured to:
identify a change in the physiologic impedance data indicative of risk of intradialytic hypotension; and
produce an alert regarding the risk of intradialytic hypotension in response to the identifying the change in the physiologic impedance data.

16. The system of claim 10, wherein the processing circuitry of the second device is configured to:
compare the sensor data sensed for a first hemodialysis treatment to sensor data sensed for another hemodialysis treatment; and
recommend a change in hemodialysis dosage based on the comparing of the sensor data.

17. The system of claim 10, wherein the processing circuitry of the second device is configured to:
compare the sensor data sensed for a first hemodialysis treatment to sensor data sensed for another hemodialysis treatment; and
produce an alert to stop a hemodialysis treatment based on the comparing of the sensor data.

18. An ambulatory medical device, the device comprising:
at least one physiologic sensor configured to produce a sensed physiological signal representative of physiological information of a patient; and
signal processing circuitry configured to:
identify patterns in the physiological signal consistent with hemodialysis treatments of the patient;
determine a change in the identified patterns indicative of a change in the hemodialysis treatments; and
produce an alert associated with the hemodialysis treatments in response to the determined change in the patterns of the physiological signal.

19. The device of claim 18, including:
an impedance sensor configured to produce a sensed impedance signal representative of physiological impedance of the patient; and
wherein the signal processing circuitry is configured to:
identify a change in the sensed impedance signal indicative of a change in the hemodialysis treatments; and
produce the alert using the identified change in the sensed impedance signal.

20. The device of claim 18, including:
a heart sound sensor configured to produce a heart sound signal representative of mechanical heart activity of the patient; and
wherein the signal processing circuitry is configured to:
identify a change in the heart sound signal indicative of a change in the hemodialysis treatments; and
produce the alert using the identified change in the heart sound signal.
